(19) United States
(12) Patent Application Publication
(10) Pub. No.: US 2016/0256618 A1
(43) Pub. Date: Sep. 8, 2016

(54) Vacuum Assisted Breastfeeding Device

(71) Applicant: UNIVERSITY OF CONNECTICUT, Farmington, CT (US)

(72) Inventor: Amy Embleton, Seattle, WA (US)

(73) Assignee: University of Connecticut, Farmington, CT (US)

(21) Appl. No.: 15/032,561

(22) PCT Filed: Oct. 28, 2014
(86) PCT No.: PCT/US14/62612

§ 371 Date: Apr. 27, 2016

Related U.S. Application Data
(60) Provisional application No. 61/896,288, filed on Oct. 28, 2013.

(51) Int. Cl. A61M 1/06

(52) U.S. Cl. CPC A61M 1/066

(57) ABSTRACT

The present disclosure provides advantageous breastfeeding devices and/or assemblies. More particularly, the present disclosure provides advantageous vacuum assisted breastfeeding devices/assemblies, and related methods of use. The present disclosure provides assemblies/devices to assist mothers in breastfeeding infants. The advantageous assemblies/devices of the present disclosure can provide feeding assistance and improved breastfeeding rates to infants (e.g., premature infants) while they are being held close to the mother and feeding. Premature infants, and full-term infants with developmental delays, transient breastfeeding problems, and or cleft lip and palate can get several benefits from the assemblies/devices of the present disclosure.

### CROSS-REFERENCE TO RELATED APPLICATIONS

This application claims the benefit of U.S. Provisional Application No. 61/896,288 filed Oct. 28, 2013, all of which is herein incorporated by reference in its entirety.

### BACKGROUND

1. Technical Field

The present disclosure relates to breastfeeding devices/assemblies and, more particularly, to vacuum assisted breastfeeding devices/assemblies and related methods of use.

2. Background Art

In general, breastfeeding devices/assemblies or the like are known. Some exemplary breastfeeding devices/assemblies and related methods/accessories or the like are described and disclosed in the Importance of Vacuum For Breastmilk Expression, (Kent et al., Breastfeeding Medicine, Vol. 3, No. 1, 11-19, 2008); Tongue Movement And Intra-Oral Vacuum In Breastfeeding Infants, (Geddes et al., Early Human Development, 84, 471-477, 2008) and Oral Feeding In Low Birth Weight Infants, (Lau et al., The Journal Of Pediatrics, Vol. 130(4), April, 1997, 561-569, 1997), and U.S. Pat. Nos. 6,676,631; D417,735; 6,663,587; 5,474,193 and 5,941,847, and WO-2010/096547, the entire contents of each being hereby incorporated by reference in their entireties.

In 2007, about 4,000,000 babies were born in the United States. About 75% of those babies (about 3,000,000) were initially breastfed. At six months the number decreased to approximately 1,800,000, and at one year decreased to approximately 1,000,000.

In general, the majority of preterm infants working on establishing full breastfeeding do partial breastfeeding, removing what milk they can from the breast, and then bottle feed or receive a tube feeding to take in enough milk for growth and nutrition. The mother, following a partial breastfeeding, needs to pump her breasts to remove the remaining milk that the infant did not consume to ensure the continuation of an adequate milk supply (supply and demand), and to collect milk for the bottle and/or tube feedings. For these infants and mothers, breastfeeding becomes a tiring three-part endeavor of breastfeeding, followed by tube or bottle feeding, and lastly breast pumping.

There are a few assistive devices that attempt to help infants to transfer/consume more milk when feeding from the breast: a nipple shield and a supplemental nursing system (SNS). In general, a nipple shield is a circular, thin silicone device with a protrusion in the center that has holes (e.g., four holes) that allow for milk flow from the mother's breast to the infant's mouth. The nipple shield is placed over a mother's nipple during breastfeeding. The infant latches onto the nipple shield that is centered over the mother's nipple and sucks/feeds. Ideally, the mother's nipple is pulled into the protruded area of the nipple shield. Milk is released into the nipple shield, and passes through the holes, and into the infant's mouth. The nipple shield has been shown to improve milk intake in preterm infants, but the mechanism for why this happens has generally not been determined. Furthermore, while a nipple shield may assist a preterm infant to remove more milk from the breast than breastfeeding alone, a nipple shield does not typically immediately enable a preterm infant to remove a full feeding volume from the breast. The infants who are capable of removing only partial feeding volumes at the breast with the nipple shield typically need to be supplemented by bottle or tube to receive adequate nutrition.

The SNS is a feeding device that consists of a reservoir/milk container, and tubing that is connected to the reservoir and taped to the mother's breast. The infant latches onto the breast, taking in both the mother's nipple and the SNS tubing. The infant receives some milk from the SNS reservoir as well as whatever milk the infant can remove from the breast. An SNS can also be used in combination with a nipple shield. In general, the SNS device is infrequently used as many mothers find it cumbersome and opt not to use this device/method. Mothers who use the SNS typically would still be required to pump to maintain an adequate supply of milk. A mother of a preterm infant generally would not be discharged to home using the SNS as an aid to full breastfeeding.

Thus, despite efforts to date, a need remains for improved breastfeeding assemblies. These and other inefficiencies and opportunities for improvement are addressed and/or overcome by the assemblies, systems and methods of the present disclosure.

### SUMMARY

The present disclosure provides advantageous breastfeeding devices and/or assemblies. More particularly, the present disclosure provides advantageous vacuum assisted breastfeeding devices/assemblies, and related methods of use.

Of note, a feature unique to these devices/assemblies when compared to conventional technologies, is that the devices of the present disclosure can remedy the specific underlying etiology of inadequate or low oral vacuum pressure that is responsible for the poor milk transfer from the breast seen in the intended population of infant users.

In exemplary embodiments, the present disclosure provides assemblies/devices to assist mothers to breastfeed their infants (e.g., feeding assistance to premature infants while being held close to the mother). In certain embodiments, preterm infants, or infants with developmental delay, or other conditions such as but not limited to cleft lip and or palate, can get several benefits from the assemblies/devices of the present disclosure.

The present disclosure provides for a breastfeeding assembly including a breastshield member configured and dimensioned to be positioned over a breast; a nipple member configured and dimensioned to be mounted with respect to the breastshield member; and a suction/vacuum source operatively connected to the breastshield member, the suction/vacuum source configured to provide vacuum or negative pressure to the maternal nipple through the breastshield member to facilitate the removal of milk for infant consumption from the breast during real-time breastfeeding.

The present disclosure also provides for a breastfeeding assembly wherein the suction/vacuum source is electric or manual. The present disclosure also provides for a breastfeeding assembly wherein the nipple member is fabricated from a food grade polymer.

The present disclosure also provides for a breastfeeding assembly wherein the nipple member includes an attachment area for the infant's mouth that includes one or more openings, slits or holes for milk to flow therethrough. The present disclosure also provides for a breastfeeding assembly wherein the suction/vacuum source is external to the breastshield member and is in communication with the breastshield member via tubing. The present disclosure also provides for a breastfeeding assembly wherein the breastshield member includes one or more milk exit portals.

The present disclosure also provides for a breastfeeding assembly further including a flow control member operatively associated with the suction/vacuum source. The present disclosure also provides for a breastfeeding assembly wherein the tubing is configured and dimensioned to attach to a breast pump. The present disclosure also provides for a breastfeeding assembly wherein a distal end of the breastshield member is configured and dimensioned to allow the maternal nipple to move in a back and forth manner relative to the distal end of the breastshield member.

The present disclosure also provides for a breastfeeding assembly wherein the suction/vacuum source is configured to regulate and facilitate the control of milk flow. The present disclosure also provides for a breastfeeding assembly wherein the suction/vacuum source is configured to be turned on and off to regulate and facilitate the control of milk flow.

The present disclosure also provides for a breastfeeding assembly wherein at least a portion of the breastshield member defines a vacuum compartment and at least a portion of the nipple member defines a milk reservoir compartment; and wherein at least one valve member is positioned between the vacuum compartment and the milk reservoir compartment, the at least one valve member configured to intermittently seal the vacuum compartment to create vacuum that intermittently elongates the maternal nipple to release milk, and then allows milk to flow from the vacuum compartment to the milk reservoir compartment and not to the tubing.

The present disclosure also provides for a breastfeeding assembly wherein the at least one valve member is a one-way valve or a check valve positioned on a supporting surface of the breastshield member, the at least one valve member configured to provide for: (i) the substantially complete and intermittent sealing of the vacuum compartment, and (ii) the flow of milk from the vacuum compartment to the milk reservoir compartment and to the infant's mouth or to a collection reservoir.

The present disclosure also provides for a breastfeeding assembly wherein at least a portion of the tubing is positioned between the breastshield member and the nipple member.

The present disclosure also provides for a breastfeeding assembly further including a connector member, the connector member configured to releasably secure the breastshield member and the nipple member relative to one another. The present disclosure also provides for a breastfeeding assembly further including a securement member, the securement member and the connector member configured to releasably secure the breastshield member and the nipple member relative to one another.

The present disclosure also provides for a breastfeeding assembly wherein the breastshield member and the nipple member are fabricated from food grade washable material. The present disclosure also provides for a breastfeeding assembly wherein the breastshield member can be fabricated and user-selected in various sizes for maternal comfort.

The present disclosure also provides for a breastfeeding assembly wherein the nipple member can be fabricated and user-selected with various levels of rigidity to provide different milk flow rates to match infant feeding ability.

The present disclosure also provides for a breastfeeding assembly wherein the nipple member can be fabricated and user-selected with various sizes or shapes of the one or more openings, slits or holes to provide different milk flow rates to match infant feeding ability. The present disclosure also provides for a breastfeeding assembly, wherein a tube connects the milk reservoir compartment to an isolated collection reservoir, the tube configured to collect milk overflow from the milk reservoir compartment and send it to the isolated collection reservoir for subsequent infant consumption.

The present disclosure also provides for a method for breastfeeding including providing a breastshield member; mounting a nipple member with respect to the breastshield member; operatively connecting a suction/vacuum source to the breastshield member; positioning the breastshield member over a breast; and providing vacuum or negative pressure via the suction/vacuum source to a maternal nipple of the breast through the breastshield member to facilitate the removal of milk for infant consumption from the breast during real-time breastfeeding.

Any combination or permutation of embodiments is envisioned. Additional advantageous features, functions and applications of the disclosed assemblies, systems and methods of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various features, steps and combinations of features/steps described below and illustrated in the figures can be arranged and organized differently to result in embodiments which are still within the spirit and scope of the present disclosure. To assist those of ordinary skill in the art in making and using the disclosed systems, assemblies and methods, reference is made to the appended figures, wherein:

FIGS. 1-2 are side views of a breastfeeding assembly according to exemplary embodiments of the present disclosure;

FIG. 3 is a partial side view of an exemplary inner breastshield according to the present disclosure;

FIG. 4 is a partial side view of an exemplary infant teat/nipple member according to the present disclosure;

FIGS. 5-6 are side views of an exemplary breastfeeding assembly according to the present disclosure;

FIGS. 7-8 are side views of an exemplary breastfeeding assembly according to the present disclosure;

FIG. 9 is a side view of an exemplary breastfeeding assembly according to the present disclosure;

FIG. 10 is a cross-sectional side view of the breastfeeding assembly of FIG. 9;

FIG. 11 is a side perspective view of an infant nipple member of the breastfeeding assembly of FIG. 9;

FIG. 12 is a side perspective view of a securement member of the breastfeeding assembly of FIG. 9;

FIG. 13 is a front-side perspective view of an outer breastshield member and potential valve site of the breastfeeding assembly of FIG. 9;

FIG. 14 is a rear-side perspective view of the outer breastshield member and potential valve site of FIG. 13;

FIG. 15 is a side perspective view of an intermediate breastshield member with a potential valve supporting surface of the breastfeeding assembly of FIG. 9; and

FIGS. 16-18 are side views of an exemplary breastfeeding assembly with a potential milk storage reservoir according to the present disclosure.

### DETAILED DESCRIPTION

The exemplary embodiments disclosed herein are illustrative of advantageous breastfeeding assemblies and systems of the present disclosure and methods/techniques thereof. It should be understood, however, that the disclosed embodiments are merely exemplary of the present disclosure, which may be embodied in various forms. Therefore, details disclosed herein with reference to exemplary breastfeeding assemblies/systems and associated methods/techniques of assembly and use are not to be interpreted as limiting, but merely as the basis for teaching one skilled in the art of how to make and use the advantageous breastfeeding assemblies/systems and/or alternative breastfeeding assemblies of the present disclosure.

The present disclosure provides improved breastfeeding devices and/or assemblies. More particularly, the present disclosure provides improved vacuum assisted breastfeeding devices/assemblies, and related methods of use.

In general, the present disclosure provides assemblies/devices to assist mothers in breastfeeding infants. For example, the advantageous assemblies/devices of the present disclosure can provide feeding assistance to infants (e.g., premature infants) during breastfeeding while the infant is being held close to the mother.

It is noted that full-term birth is generally defined as 37 to 40 weeks of pregnancy. Premature birth is generally defined as birth occurring at 36 weeks of pregnancy or less. In exemplary embodiments, preterm infants, or developmentally delayed infants, can get several advantageous benefits from the assemblies/devices of the present disclosure.

In certain embodiments, the present disclosure provides for an assistive assembly/device for lactating mothers to use to breastfeed infants that have sucking and/or other feeding problems that prevent the infant from adequately removing enough milk directly from the breast to sustain growth and nutrition. In general, the exemplary assembly/device provides an external suction/vacuum source applied to the nipple to aid the infant in stimulating the milk ejection reflex, and subsequently removing milk from the breast.

One target infant population for this assembly is preterm infants, including the growing sub-group of late preterm infants. The assembly could also be used with other infants, e.g., full-term infants with transient sucking and latch problems, infants with neurological problems, and/or infants with cleft lip and/or palate.

The benefits of breastfeeding for infants, including those born prematurely, are well documented. Major worldwide public health initiatives aim to improve both breastfeeding initiation rates and duration, though many preterm infants are not breastfed. The recommendation to breastfeed is endorsed by the American Academy of Pediatrics, the U.S. Surgeon General's Office and the World Health Organization.

One problem with preterm infants, with regard to breastfeeding, is that effective breastfeeding typically requires a mature feeding pattern. A mature, fully developed feeding pattern typically consists of strong enough suction pressure that alternates with compression pressure applied to the maternal nipple to elicit the milk ejection reflex. This reflex makes milk available for removal by the infant. Healthy, full-term infants are generally born with this type of feeding pattern. Preterm infants typically do not develop a strong enough suction component to successfully remove adequate milk from the breast until they reach full-term corrected age (what their full-term due date was calculated to be) or for many, not until they are 2-4 weeks corrected age (past what their due date was). In general, it is a developmental feeding issue, an issue of maturity that is the underlying etiology for low milk removal, and therefore sub-optimal breastfeeding in preterm infants.

Until a preterm infant develops a mature feeding pattern that generates enough suction/vacuum, typically he or she is unable to remove full feeding volumes from the breast, and is at risk for being under fed if exclusively breastfed. Infants with clefts of the palate, due to the anatomical defect and subsequent abnormal opening in the oral cavity, are almost never able to generate enough vacuum to breastfeed until they are repaired surgically. Clefts of the palate are typically not corrected surgically until about four months of age, and often require multiple surgeries over time.

In almost all preterm infants in the newborn intensive care nurseries, mastery of bottle feeding is observed at a much earlier age than mastery of breastfeeding. Preterm infants are capable of beginning to take full feeding volumes from a bottle beginning around 34 weeks corrected age. It is documented in the preterm infant feeding literature that the earliest feeding patterns, and the feeding patterns present when preterm infants are most likely to begin oral feeding, demonstrate a compression-only or a predominantly compression feeding pattern, without or with low amplitude and irregular frequency of suction/vacuum pressure. Furthermore, the lactation literature has documented that the milk ejection reflex is dependent on adequate vacuum pressure.

Mothers of the above described premature infants are typically required to use a breast pump to maintain their milk supply until their infant is capable of full breastfeeding. Over time, the pump does not seem to be as effective as an infant feeding at the breast at maintaining a mother's milk supply. As the earliest age of viability for preterm infants is currently 23 weeks of gestation, some mothers could conceivably need to pump their milk for up to 21 weeks before their baby can fully breastfeed. A high number of these pump-dependent mothers will eventually develop low milk supply issues, and/or perceive their required breast pumping frequency schedule as too difficult. These mothers often give up on breastfeeding before their infants are developmentally capable of exclusive breastfeeding. From a public health standpoint, this is sub-optimal nutrition and feeding.

Current practice provides that conventional devices do not enable infants who are unable to remove a full feeding volume of milk from the breast, to remove enough milk to sustain growth and nutrition without the need for additional tube or bottle feeding.

In exemplary embodiments, the present disclosure provides for an advantageous breastfeeding assembly that includes an external vacuum source applied to the mother's nipple during real-time breastfeeding, thereby providing a significant operational (e.g., breastfeeding), commercial and/or manufacturing advantage as a result. Additionally, the present disclosure provides for an advantageous breastfeeding assembly that is compatible with commercial breast pumps, thereby providing a significant operational, commercial and/or manufacturing advantage as a result.

In certain embodiments, the breastfeeding assembly includes a double chamber flange system: (i) a maternal chamber, and (ii) a milk collection/infant chamber. The maternal chamber is configured and dimensioned to provide vacuum assisted breast pumping and expression of breast milk. The milk collection/infant chamber is configured and dimensioned to provide a reservoir for the collection and availability of milk, and to provide a point of attachment for the infant's mouth for sucking and feeding.

In exemplary embodiments, the vacuum source is provided via a thin tube that is compatible with commercial breast pumps (e.g., with the vacuum lines of commercial breast pumps). In some embodiments, the breastfeeding assembly provides vacuum only to the maternal chamber, and allows for the flow of milk from the maternal chamber to the infant chamber.

In certain embodiments, the breastfeeding assembly design includes a low profile to allow for close proximity between mother and infant as is normal in typical breastfeeding. In accordance with the current standard practice for safe breastmilk collection and infant feeding, exemplary materials to be used will be dishwasher safe and/or cleanable with dishwashing soap and warm water.

In exemplary embodiments, the breastfeeding assembly provides one or more of the following: (i) low profile—to keep the baby in close proximity to mother; (ii) milk flow is physically separated from the vacuum supply; (iii) milk can flow from the device directly into the infant's mouth and overflow can be collected in a reservoir/bottle; (iv) milk flow rate can be regulated; (v) breastshields can be made in various dimensions for maternal comfort; (vi) the design is compatible with the vacuum supply from commercial breast pumps; (vii) the design can be fabricated from materials which are dishwasher safe or can be cleaned with soap and water (moreover, if a membrane is used in the assembly/design, it can be disposable); and/or (viii) one or more valves may be used to: (1) seal the maternal chamber to apply vacuum to the nipple and allow for the flow of milk into the infant chamber, (2) to reduce the amount of compression required for the infant to remove milk from the infant chamber when sucking.

In general, the breastfeeding assembly is fairly compact, easy to use, comfortable, a non-formidable barrier between mother and child, and/or perceived as an assembly acceptable to mothers. Moreover, the breastfeeding assembly can be fabricated from materials approved for contact with food and/or amenable to injection molding. Further, the assembly materials can be fabricated from materials that can be sterilized or cleaned according to accepted standards and practices in infant feeding, and for the safe collection and handling of human milk.

### Exemplary Breastfeeding Assembly Attributes:

In exemplary embodiments, the breastfeeding assembly (e.g., double chamber assembly) is easy to clean. In some embodiments and as further discussed below, the assembly is secured (e.g., screwed) together using a securement member (e.g., outer ring member) to secure the parts together. Moreover, the assembly can be low profile. The vacuum can be applied through an annulus anterior to the mother's nipple.

In exemplary embodiments, the vacuum is isolated from the milk flow (e.g., by a membrane). The length of the narrow end of the breastshield can be stepped down to the size of conventional/commercially available breastshields. In one embodiment, the length of the volume that accommodates the expansion of the mother's nipple in the breastshield is approximately 7/8″. This configuration has been found to be sufficient to allow for the stimulation and stretching of the maternal nipple, and for the subsequent expression of milk.

In certain embodiments, the diameter of the narrow end of the breastshield is about 24 mm or about 27 mm. In general, the compartment on which the baby will latch onto and suckle from is typically small. Typically, a smaller compartment/volume space requires less compression or sucking effort from the infant to accomplish the expression/removal of milk for feeding.

The outer surface of the breastshield can be ridged to prevent collapse of the outer compartment which may restrict milk flow. If gravity is desired/required to get milk to flow to the second compartment, an off-center feeding compartment can be provided. However, on axis feeding is typically preferred.

Information about typical vacuum cycling (e.g., period and pressure) is described and disclosed in U.S. Pat. No. 6,676,631, the entire contents of which is hereby incorporated by reference in its entirety.

In exemplary embodiments, if milk will also be collected while the infant is feeding, then the device can prevent and assure there is substantially no contamination of the collected milk that can be used for future feeding (e.g., this can be accomplished through a back flow preventer or the like).

Referring now to the drawings, like parts are marked throughout the specification and drawings with the same reference numerals, respectively. Drawing figures are not necessarily to scale and in certain views, parts may have been exaggerated for purposes of clarity.

With reference to FIGS. 1-2, there is illustrated an exemplary breastfeeding assembly 10 according to an embodiment of the present disclosure. In general, breastfeeding assembly 10 is configured and dimensioned to be utilized for breastfeeding or the like.

Breastfeeding assembly 10 advantageously enables infants (e.g., infants who are unable to remove a full feeding volume of milk from the breast) to remove enough milk to sustain growth and nutrition without the need for additional tube or bottle feeding. Assembly 10 can also reduce the steps required in the feeding sequence described above, as it can eliminate the need, or some of the need for pumping after breastfeeding.

One advantageous feature of exemplary assembly 10 is that it provides an (external) suction/vacuum source 12 (e.g., external breast pump 12) to the mother's nipple during real-time breastfeeding (e.g., via line or tubing 14). In exemplary embodiments, the suction/vacuum source 12 (e.g., external breast pump 12) drives the let-down reflex and allows the milk to easily flow from the breast, and thereby compensates for the low vacuum pressures that are developmentally normal, and typically seen in an immature infant's feeding pattern, as well as in the feeding pattern of full-term infants with breastfeeding problems due to various etiology as previously described.

Some literature on breastfeeding implicates the vacuum component of infant sucking as a major factor in milk removal from the breast. One breast pump study showed that maximum comfortable vacuum levels resulted in significantly more milk versus lower vacuum levels. Currently, there is no conventional device that definitely and specifically compensates for the low intra-oral vacuum pressures seen in immature infants and full-term infants with sucking problems.

In certain embodiments and as shown in FIGS. 1-2, a breastshield 16 and nipple member 20 are configured and dimensioned to be positioned over the lactating breast/nipple that the baby latches onto. The nipple member 20 can have an exit portal allowing milk to flow into the baby's mouth. A small tube 14 can be connected to the breastshield compartment 16 to apply vacuum into the breastshield 16 and to the maternal nipple to drive the milk ejection reflex, and subsequently deliver adequate volumes of milk to the infant. Tube 14 can be attached to the breastshield 16 in such a way that it will not come into contact with the baby's mouth.

In certain embodiments, assembly 10 includes separate compartments for the application of the vacuum and for the collection of the milk, with a valve member 18 (e.g., one-way valve, check-valve, diaphragm check valve, clack valve, non-return valve or the like) that allows the milk to flow from the vacuum/maternal breastshield compartment to the milk reservoir/infant nipple compartment. As such, assembly 10 can include a (small) milk collection reservoir/infant teat, to hold milk once expressed from the breast for infant consumption.

One advantageous feature of assembly 10 can be a flow rate control member, as preterm infants often have trouble feeding if they receive milk too fast. For example, the flow rate feature/member can be self-paced technology—driven by the baby's sucking pattern and flowing only when the infant is sucking, or the flow rate feature/member could have an easy to use flow control switch or the like that the mother/user could operate. In certain embodiments, the flow control switch is easily activated (e.g., by the mother's hand, foot, etc.). Likewise, in certain embodiments, the nipple member 20 can be made with different nipple flow rates by changing the hole size, shape, and/or the rigidity of the material used (e.g., silicone material, polymer, etc.).

In exemplary embodiments, assembly 10 includes an element 12 that delivers the vacuum through tubing 14 (e.g., thin tubing 14). This element 12 can be small, compact and easy to use. It can be electric or manual, and can be pumped by the mother's foot or the like. In general, many newborn intensive care nurseries and mother's of preterm infants, as well as mothers of other subgroups of infants that have breastfeeding problems, have a breast pump 12 with tubing 14 for milk expression. The tubing 14 for assembly 10 can be universally sized and fabricated to attach to conventional breast pumps or the like.

It is noted that it is not required to have tubing 14 connected to the breastshield 16 (or other sections of assembly 10) to create suction. For example, a small pump 12 can be directly connected to the breastshield 16 with no tubing 14. As such, a pump 12 or the like can be directly attached to assembly 10, thereby eliminating and/or reducing the need for tubing 14 in between the assembly 10 and the pump 12.

In other embodiments, it is noted that the pulling of breast milk does not have to be via suction (e.g., via a pump 12). The device 10/reservoir can still work if there is hand expression or some other external compression (e.g., a contracting band squeezing around the breast). In certain embodiments, the expression of milk is produced by an external pressure on or around the breast (e.g., a contracting band, hand expression or rolling mechanism), thereby eliminating the need for an internal pulling suction force and the pump. A variety of methods and mechanisms can be used to contract the band or roller, such as a pump, motor-mechanism, pistons, etc.

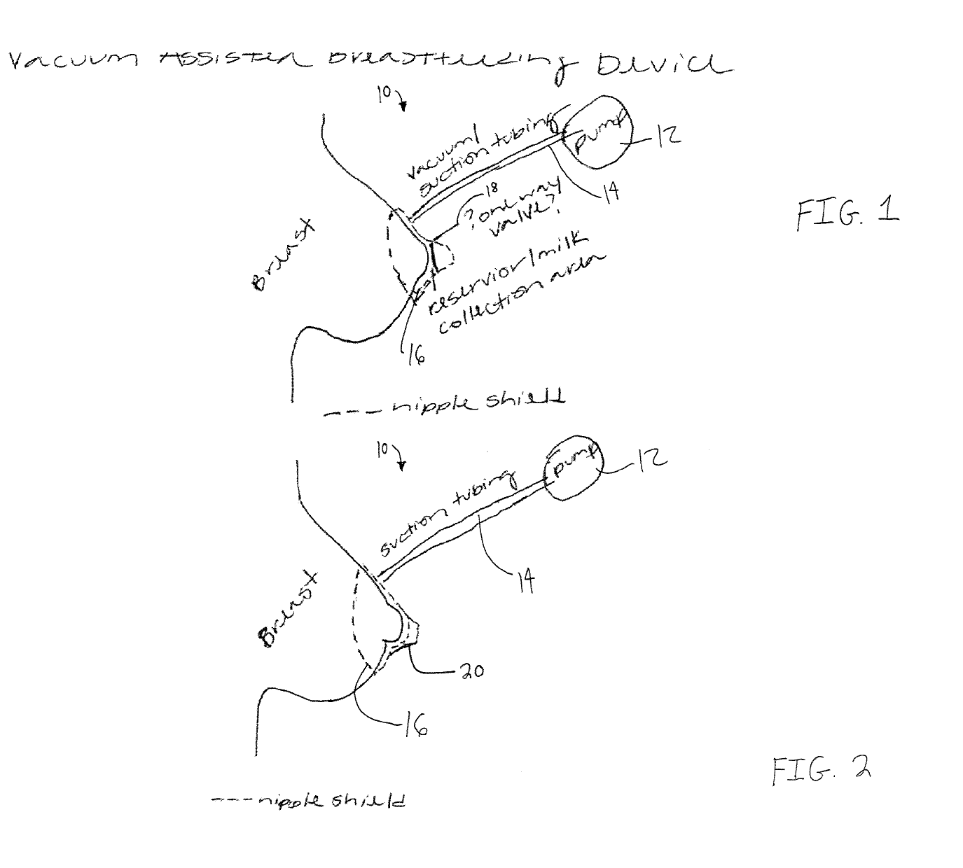

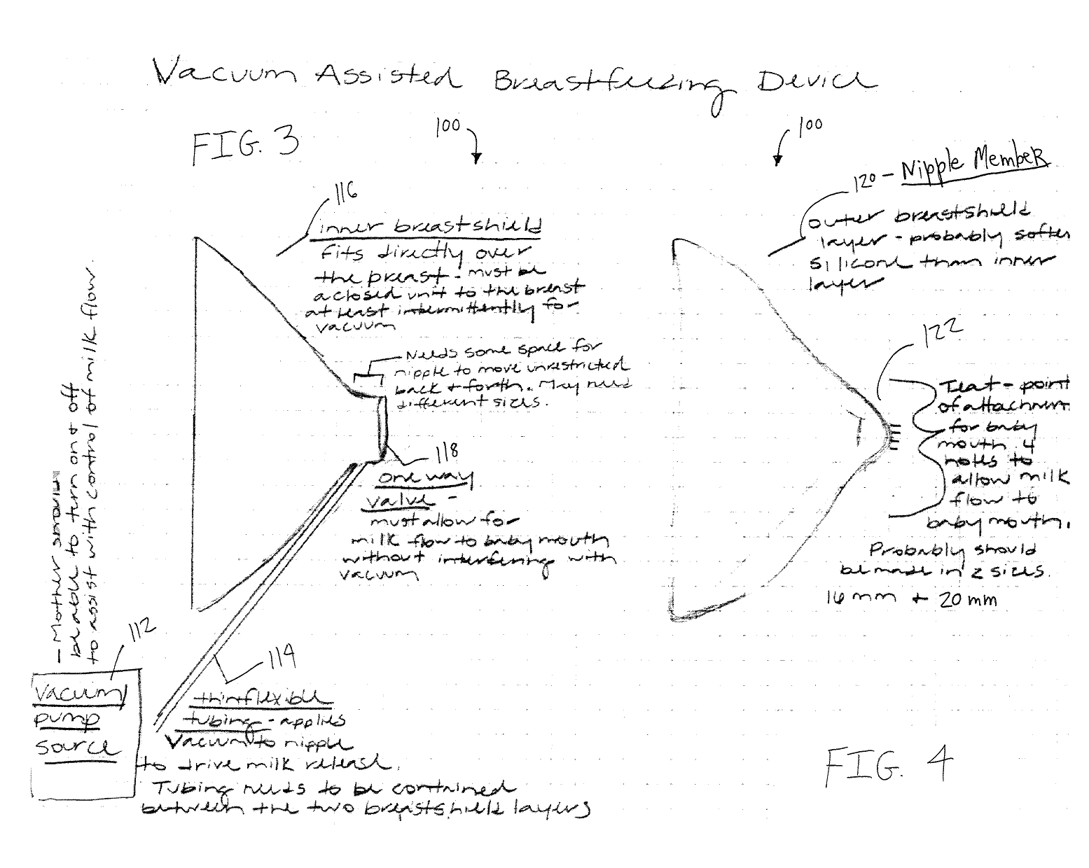

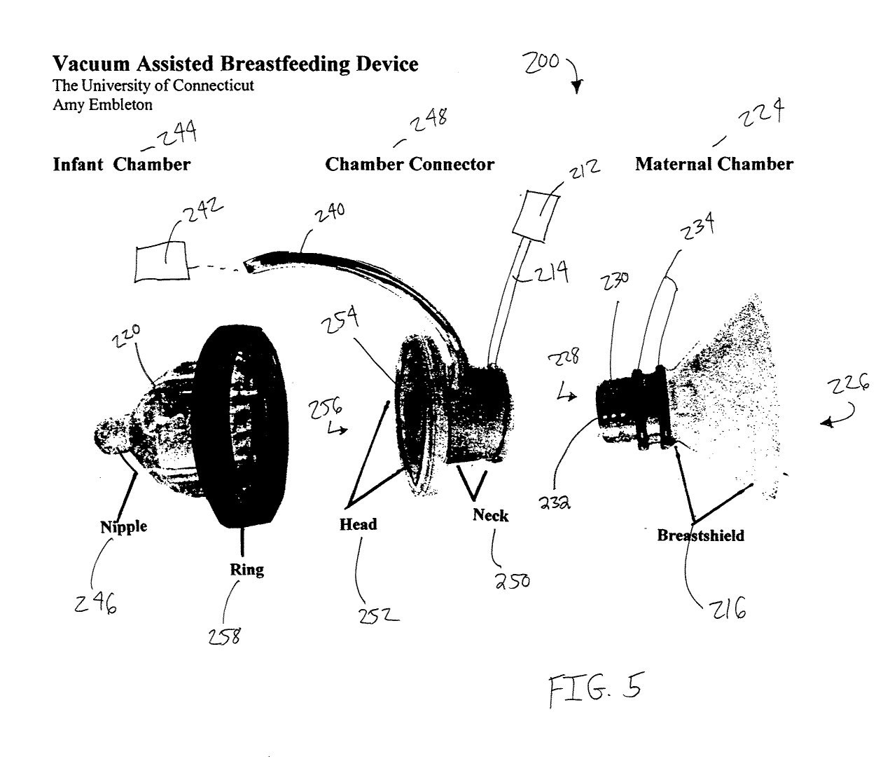

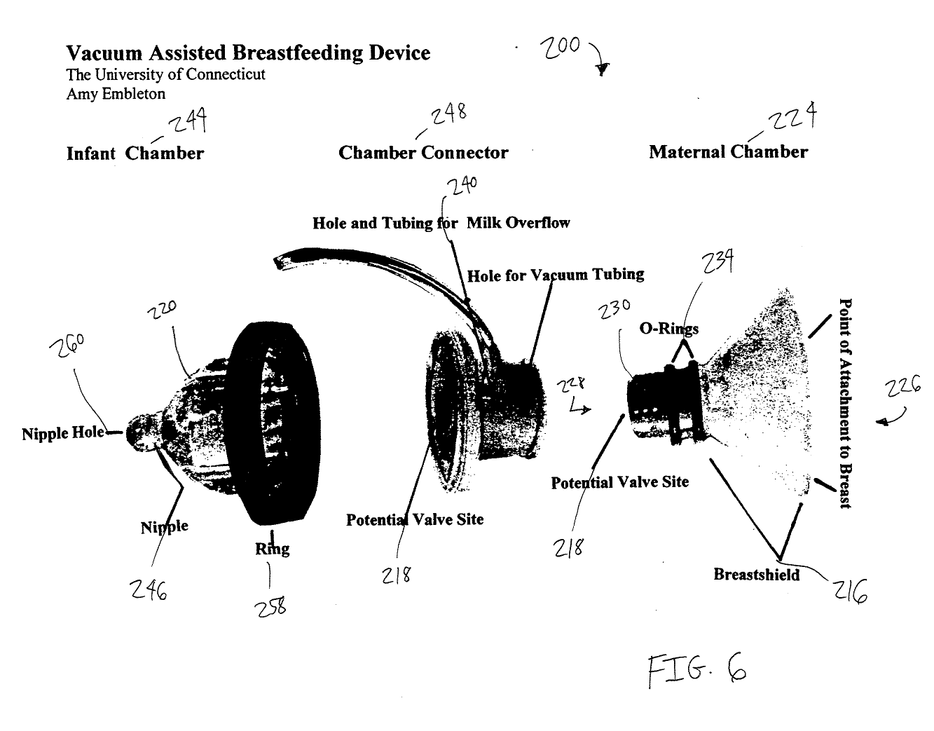

With reference to FIGS. 3-4, there is illustrated another exemplary breastfeeding assembly 100 according to an embodiment of the present disclosure. Breastfeeding assembly 100 is configured and dimensioned to be utilized for breastfeeding or the like. In general, assembly 100 is structurally and/or functionally similar to assembly 10 discussed above, with some differences.

In certain embodiments and as shown in FIGS. 3-4, a breastshield 116 (e.g., an inner breastshield) is configured and dimensioned to be positioned over the lactating breast/nipple. The breastshield 116 can have an exit portal allowing milk to flow therethrough. In exemplary embodiments, the distal end of breastshield 116 provides some space for the nipple to move unrestricted back and forth.

A small tube 114 can be connected to the breastshield 116 to apply vacuum (via suction/vacuum source 112 or pump 112) into the shield 116 and to the maternal nipple to drive the milk ejection reflex, and subsequently deliver adequate volumes of milk to the baby. In general, the breastshield 116 is a closed unit relative to the breast, at least intermittently, to allow for the application of the vacuum to the breast that cause the stretching and elongation of the maternal nipple. In general, the mother can turn on/off source 112 to assist with the control of milk flow.

In certain embodiments, assembly 100 includes separate compartments sealed intermittently by a valve member 118 (e.g., one-way valve, diaphragm check valve, etc.) to apply the vacuum only to the maternal nipple and also allow the milk to flow from the vacuum/maternal nipple compartment (116) to the milk reservoir/infant feeding compartment (120).

As shown in FIG. 4, assembly 100 further includes a nipple member 120. In some embodiments, nipple member 120 is fabricated from a material (e.g., from a polymer, from a food grade polymer, silicone, etc.) that is softer than the breastshield 116. In general, the nipple member 120 is configured and dimensioned to be positioned over the breastshield 116, and includes a teat-point of attachment area 122 that includes an opening, holes (e.g., four holes) or a slit to allow milk to flow to the baby's mouth. It is noted that teat-point of attachment area 122 can be various sizes (e.g., 16 mm, 20 mm, etc.). In certain embodiments, at least a portion of tubing 114 is contained/positioned between the breastshield 116 and the nipple member 120 to apply vacuum to the breast only.

As such, and in exemplary embodiments, assembly 100 allows vacuum (e.g., from standard breast pump 112) to be applied in the area of the maternal nipple to allow the nipple to be stretched/pulled back and forth into the breastshield 116, and back to its resting position. Assembly 100 also provides for a route for the milk to exit the breastshield 116, and enter the nipple member 120 and the infant's mouth, and not into the vacuum tubing 114. Assembly 100 also provides for an external infant teat area 122 for the infant's mouth to latch onto, thereby allowing for the flow of milk from the breast/assembly 100 into the infant's mouth for feeding.

In regards to milk flow rate control, it is noted that an electric breast pump 112 may remove milk more quickly than an infant can consume milk. One method to combat this discrepancy is to allow the mother/user to be able to turn the breast pump 112 on/off.

In exemplary embodiments, assembly 100 and/or its components are easy to clean (e.g., with warm soapy water), especially all parts/components that contact the milk and/or the infant's mouth.

In alternative embodiments, it is noted that breastshield 116 and nipple member 120 can be integral with one another (e.g., there does not have to be two different sections). For example, assembly 100 can include a single breastshield 116 where the outside or distal end is in the shape of the nipple (e.g., because a full reservoir is not needed in the nipple).

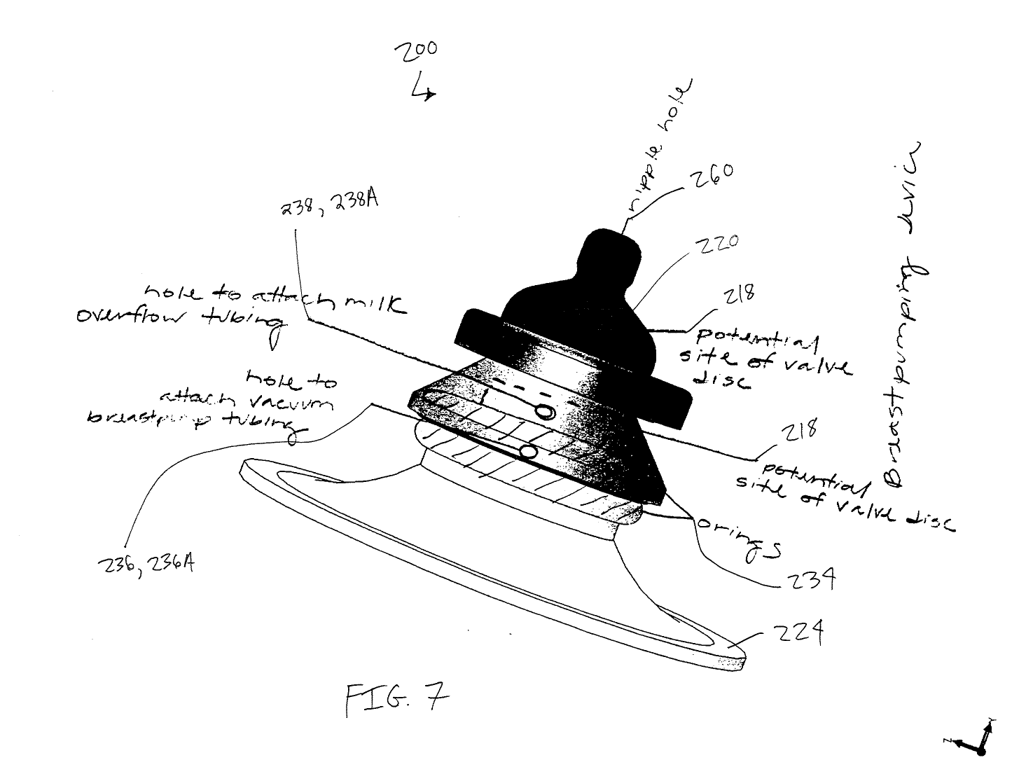

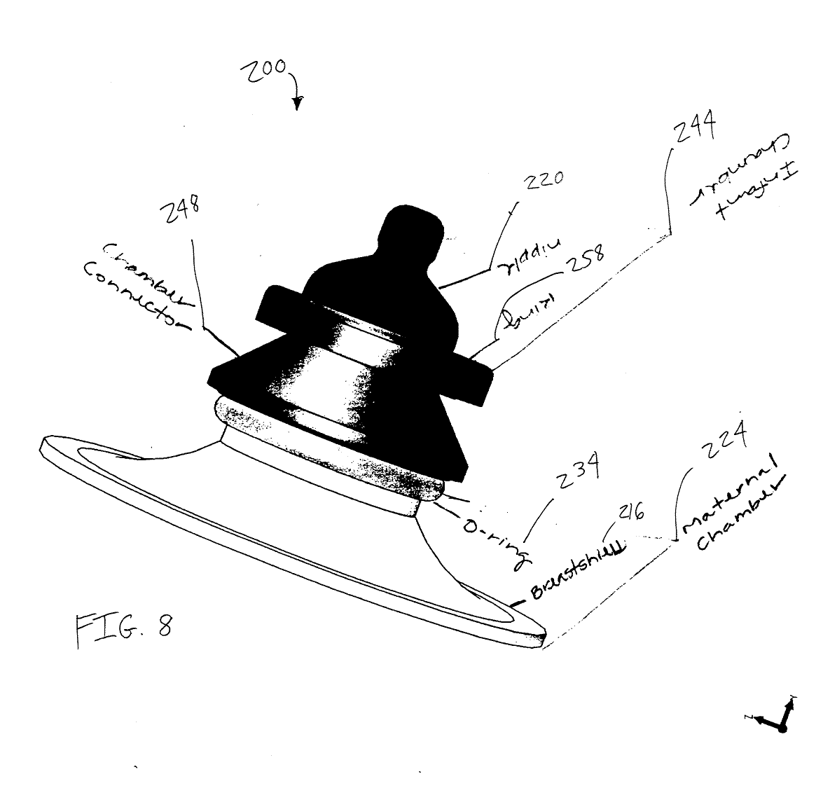

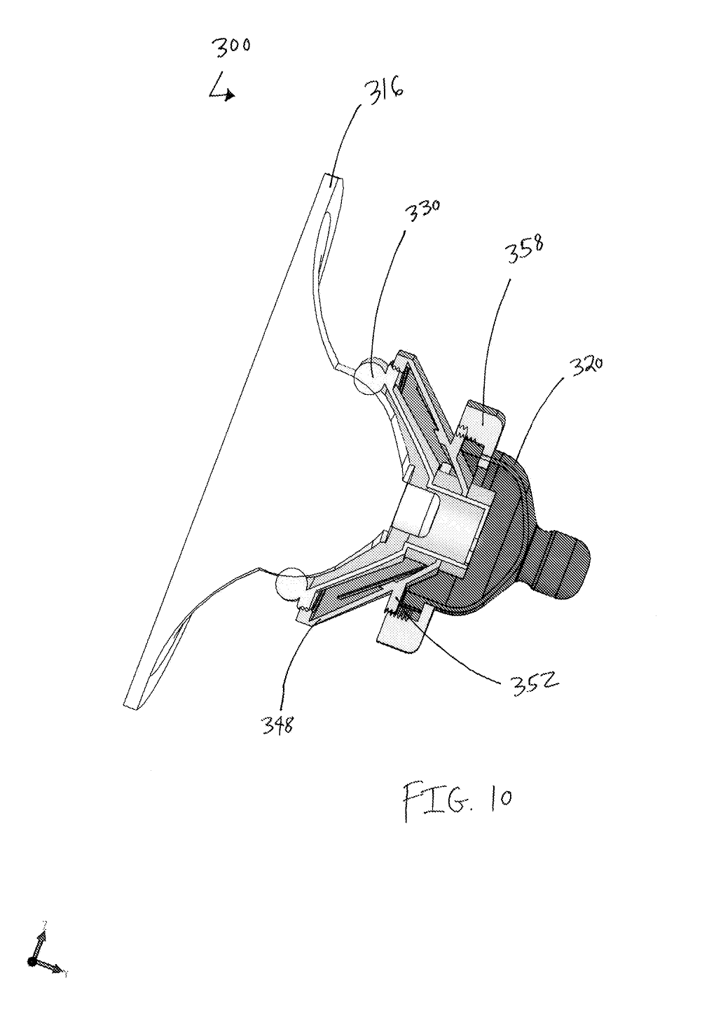

With reference to FIGS. 5-8, there is illustrated another exemplary breastfeeding assembly 200 according to an embodiment of the present disclosure. Breastfeeding assembly 200 is configured and dimensioned to be utilized for breastfeeding or the like. In general, assembly 200 is structurally and/or functionally similar to assembly 10 and/or 100 discussed above, with some differences.

### Maternal Chamber 224

In exemplary embodiments, the maternal chamber 224 includes an inner (molded) breastshield 216 (e.g., inner breast cup member 216). The proximal end 226 of the breastshield 216 is open and fits over the breast, centered over the nipple and areola. At the distal end 228 of the breastshield 216, the infant end, there is a tubular section or intermediate breastshield member 230 having an opening 232 (e.g., circular opening—obscured). In certain embodiments, the tubular section 230 accommodates the movement and elongation of the maternal nipple and areola when vacuum is applied by a suction/vacuum source 212 (e.g., breast pump 212) via line/tubing 214.

It is noted that the tubular section 230 of the breastshield 216 can be fabricated with different size (diameter) openings 232 for custom fitting for maternal comfort.

One or more gasketing members 234 (e.g., two o-rings) are seated on the outside of the tubular section 230 at the infant end 228 of the breastshield 216. In exemplary embodiments, one function of the o-rings 234 is to form a sealed annular volume where fluid cannot enter and vacuum can be pulled through to apply negative pressure to the nipple and areola to elicit the let-down reflex and the release of milk.

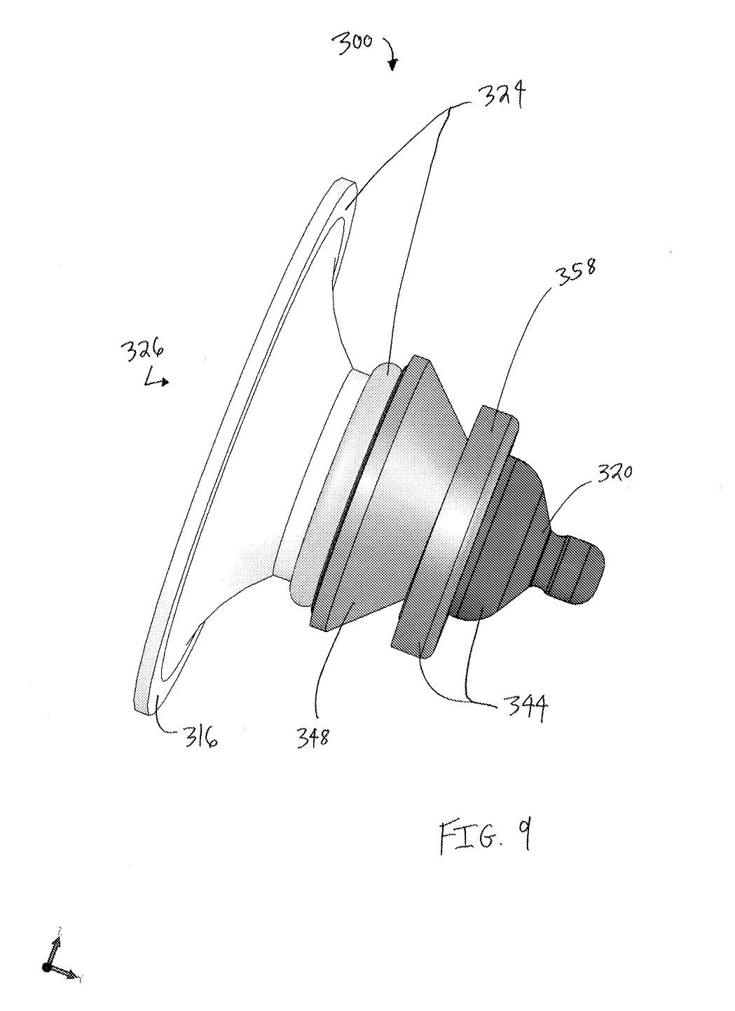

There are two holes 236, 238 (e.g., two small holes) in the distal end 228 of the maternal chamber 224 (FIG. 7). One hole 236 is located between the o-rings 234. A hose barb and tubing 214 are inserted into this hole 236 to apply vacuum to the nipple using a vacuum source 212 (e.g., breast pump 212). The second hole 238 is used to insert tubing 240 into the fluid area of the assembly 200 to evacuate breast milk to a collection reservoir 242 if the assembly 200 becomes full of breast milk. In use and in certain embodiments, the orientation of the holes 236 and/or 238 is on the top or superior aspect of the maternal chamber 224.

### Valve(s) 218

In exemplary embodiments, at least one valve member 218 (e.g., a removable, thin, one-way valve and disc/supporting surface, diaphragm check valve, etc.) allows for the anterograde flow of milk from the maternal chamber 224 to the infant chamber 244 only. When the valve 218 is open, milk evacuates the maternal chamber 224 and moves forward through the assembly 200 (e.g., towards the infant chamber 244). The closed valve 218 seals the maternal chamber 224 allowing for the breast pump 212 to apply vacuum to the breast. Vacuum pressure elongates the maternal nipple and areola to elicit the let-down reflex and the subsequent release of breast milk. As such, exemplary valve member 218 (e.g., diaphragm check valve 218) is configured to intermittently seal the vacuum compartment 224 to create vacuum that intermittently elongates the maternal nipple to release milk, and then allows milk to flow from the vacuum compartment 224 to the milk reservoir compartment 244 and not into tubing 214. The valve member 218 (e.g., valve disc/supporting surface) maintains the position of the valve 218 and allows the valve 218 to open and close while preventing the valve 218 from collapsing in a retrograde direction as pressure changes within the assembly 200.

The valve 218 also functions to seal the infant chamber 244 when the baby is sucking and allows for milk to be expressed from the infant chamber nipple 246 to the infant's mouth. It is noted that the valve 218 and supporting/attachment surface can be located either between the maternal chamber 224 and the chamber connector 248, and/or between the chamber connector 248 and the infant chamber 244. An advantage to having a valve 218 located more distally to the infant chamber 244 is to reduce the volume of the nipple in the infant chamber 244, and thereby lowering the amount of vacuum and or compression pressure required by the infant's mouth to remove milk. This feature allows for the removal of milk by infants with weak or problematic sucking ability. An alternate design includes two removable valves 218 and supporting surfaces/mechanisms, one located at each juncture discussed above.

### Chamber Connector 248

In exemplary embodiments, the chamber connector or outer breastshield member 248 (e.g., a molded part) attaches the maternal 224 and infant chambers 244, and allows for the passage of milk from the maternal 224 to the infant chamber 244. In general, the chamber connector or outer breastshield member 248 includes at least two areas—the neck 250 and the head 252.

The neck 250 fits over the distal end 228 of the maternal chamber 224. There are two small holes 236A, 238A (FIG. 7) in the neck 250. In use, the connector 248 is oriented with the holes 236A, 238A on the superior aspect of the neck 250. When the connector 248 is attached to the maternal chamber 224, the proximal hole 236A is positioned between the o-rings 234. Breast pump tubing 214 attaches to this proximal hole 236A to apply vacuum to the breast.

A tube 240 that is connected to a milk collection reservoir 242 attaches to the distal hole 238A, and collects milk overflow. In general, tube 240 connects the milk reservoir compartment 244 to the isolated collection reservoir 242, the tube 240 configured to collect milk overflow from the infant chamber/milk reservoir compartment 244 and send it to the isolated collection reservoir 242 for subsequent infant consumption.

In exemplary embodiments, the head 252 of the chamber connector 248 is substantially circular and is configured and dimensioned to attach to the infant chamber 244. The head 252 has a round opening 254 at the outer distal end 256 to allow for the flow of milk into the infant chamber 244.

### Infant Chamber 244

In exemplary embodiments, the infant chamber 244 includes an outer member 220 (e.g., a wide-base, food grade, silicone nipple member 220) and a securement member 258 (e.g., circular ring member 258). In certain embodiments, the ring 258 fits tightly over the head 252 of the chamber connector 248, and securely and releasably attaches/secures the infant chamber 244 to the chamber connector 248. The ring 258 is open and allows for milk to flow into the nipple member 220 in the infant chamber 244. It is noted that a one-way valve or the like can be placed at this site. The function of having a valve (e.g., 218) located at this site is to reduce the volume of the nipple 246 in the infant chamber 244 to lower the amount of vacuum and/or compression pressure required by the infant's mouth to remove milk.

There is at least one small opening 260 in the end of the nipple member 220 in the infant chamber 244, allowing for milk to flow out of the nipple 246 and into the infant's mouth when sucking. It is noted that the nipple member 220 in the infant chamber 244 can be made with different nipple flow rates by changing the hole size, shape, and/or the rigidity of the material (e.g., silicone material, food grade polymer, etc.).

In alternative embodiments, it is noted that an inner tube (e.g., tube 240) can be directly connected to the reservoir in the breastshield 216 (e.g., external to the valve 218). This could eliminate the need for the chamber connector 248. Alternatively and/or additionally, milk overflow can be directly from a tubing (e.g., tubing 240) directly connected to the infant chamber 244 (e.g., to outer member 220).

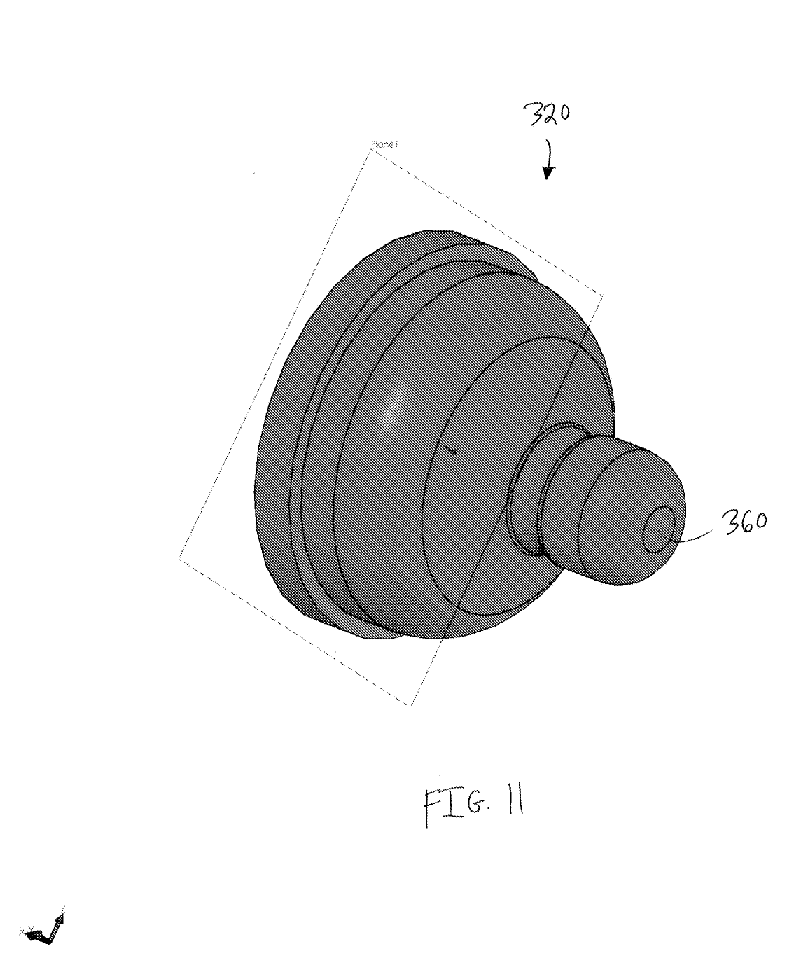

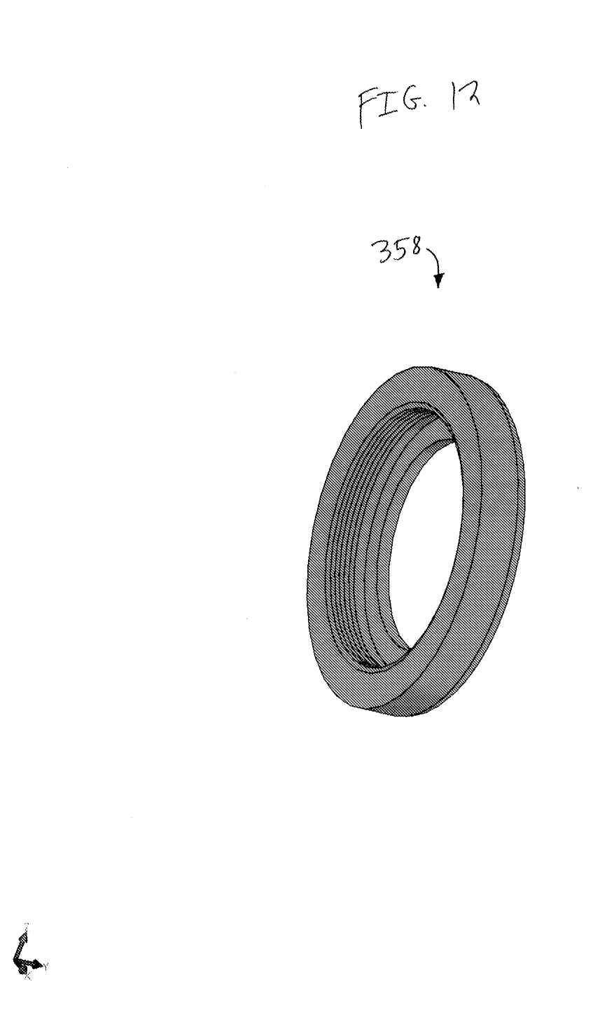

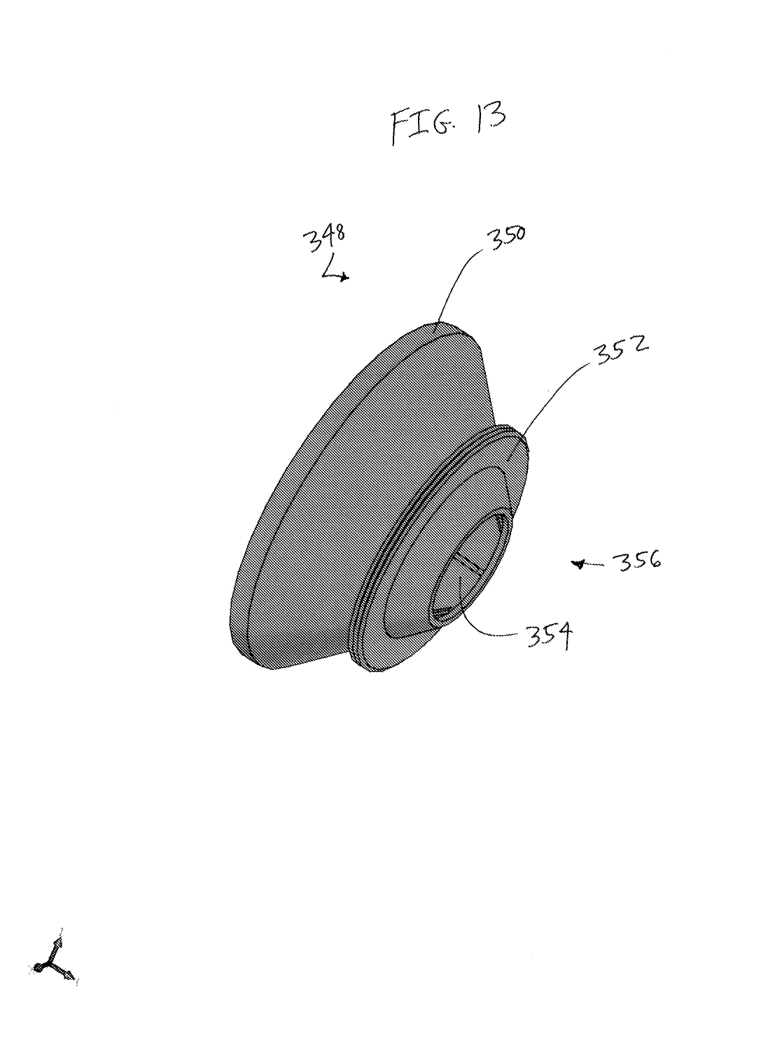

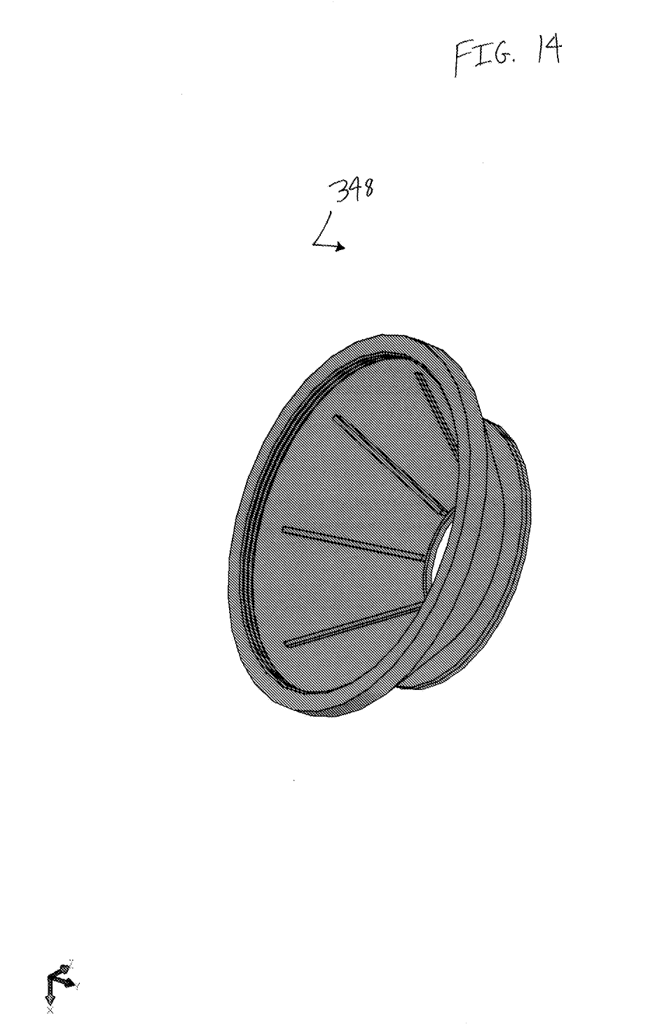

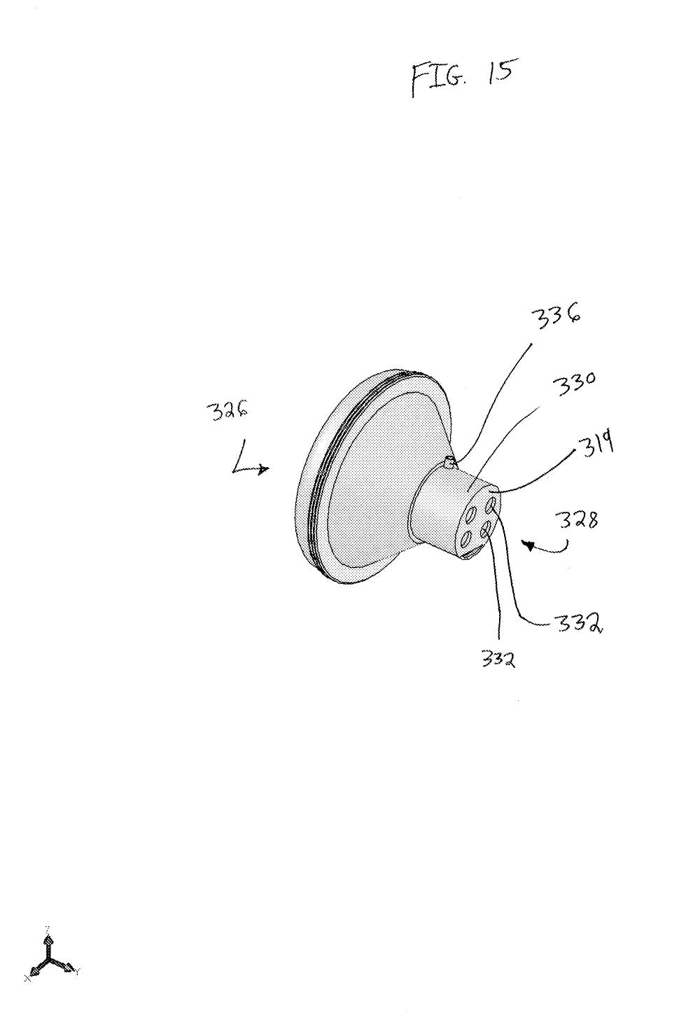

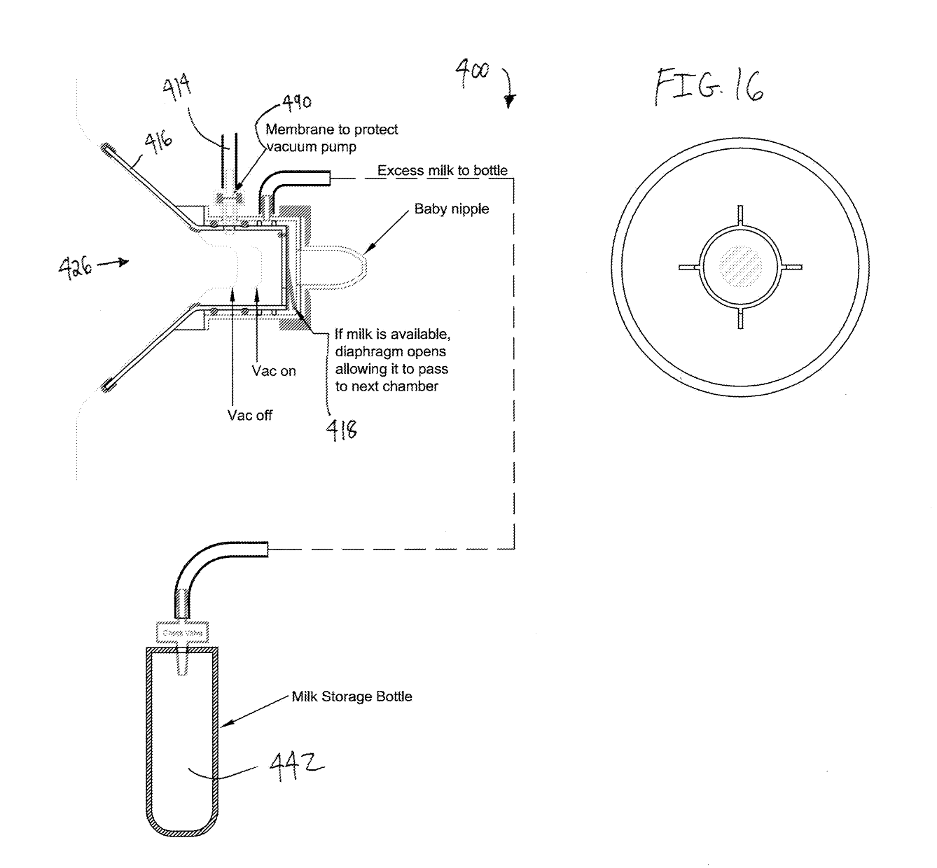

With reference to FIGS. 9-15, there is illustrated another exemplary breastfeeding assembly 300 according to an embodiment of the present disclosure. Breastfeeding assembly 300 is configured and dimensioned to be utilized for breastfeeding or the like. In general, assembly 300 is structurally and/or functionally similar to assembly 10 and/or 100/200 discussed above, with some differences.

### Maternal Chamber 324

In exemplary embodiments, the maternal chamber 324 includes an inner (molded) breastshield 316 (e.g., inner breast cup member 316). The proximal end 326 of the breastshield 316 is open and fits over the breast, centered over the nipple and areola. At the distal end of the breastshield 316, the infant end, there is a tubular section or intermediate breastshield member 330 having an opening 332 (e.g., circular opening). In certain embodiments, the tubular section or intermediate breastshield member 330 accommodates the movement and elongation of the maternal nipple and areola when vacuum is applied by a suction/vacuum source.

There is at least one hole 336 in the distal end 328. A hose barb and tubing are inserted into this hole 336 to apply vacuum to the nipple using a vacuum source. A second hole can be used to insert tubing into the fluid area of the assembly 300 to evacuate breast milk to a collection reservoir.

### Valve(s)

In exemplary embodiments, at least one valve member (e.g., similar to valve member 218) allows for the anterograde flow of milk from the maternal chamber 324 to infant chamber 344 only, as similarly described above. The valve member (e.g., valve disc and supporting/attachment surface 319) maintains the position of the valve and allows the valve to open and close while preventing the valve from collapsing in a retrograde direction as pressure changes within the assembly 300.

### Chamber Connector 348

In exemplary embodiments, the chamber connector or outer breastshield member 348 attaches the maternal 324 and infant chambers 344, and allows for the passage of milk from the maternal 324 to the infant chamber 344. In general, the chamber connector or outer breastshield member 348 includes at least two areas—the neck 350 and the head 352.

The neck 350 fits over the distal end 328 of the maternal chamber 324. There can be two small holes in the neck 350.

In exemplary embodiments, the head 352 of the outer breastshield member 348 is configured and dimensioned to attach to the infant chamber 344. The head 352 has an opening 354 at the outer distal end 356 to allow for the flow of milk into the infant chamber 344.

### Infant Chamber 344

In exemplary embodiments, the infant chamber 344 includes an outer member 320 (e.g., nipple member 320) and a securement member 358 (e.g., circular ring member 358). In certain embodiments, the ring 358 fits tightly over (e.g., threadably engaged with) the head 352 of the chamber connector 348, and securely and releasably attaches/secures the infant chamber 344 to the chamber connector 348. The distal most aspect of the ring 358 is open and allows for milk to flow into the nipple 320 in the infant chamber 344. It is noted that a one-way valve or the like can be placed at this site. The function of having a valve located at this site is to reduce the volume of the nipple 320 in the infant chamber 344 to lower the amount of vacuum and/or compression pressure required by the infant's mouth to remove milk.

There is at least one small opening 360 in the end of the nipple 320 in the infant chamber 344, allowing for milk to flow out of the nipple 320 and into the infant's mouth when sucking.

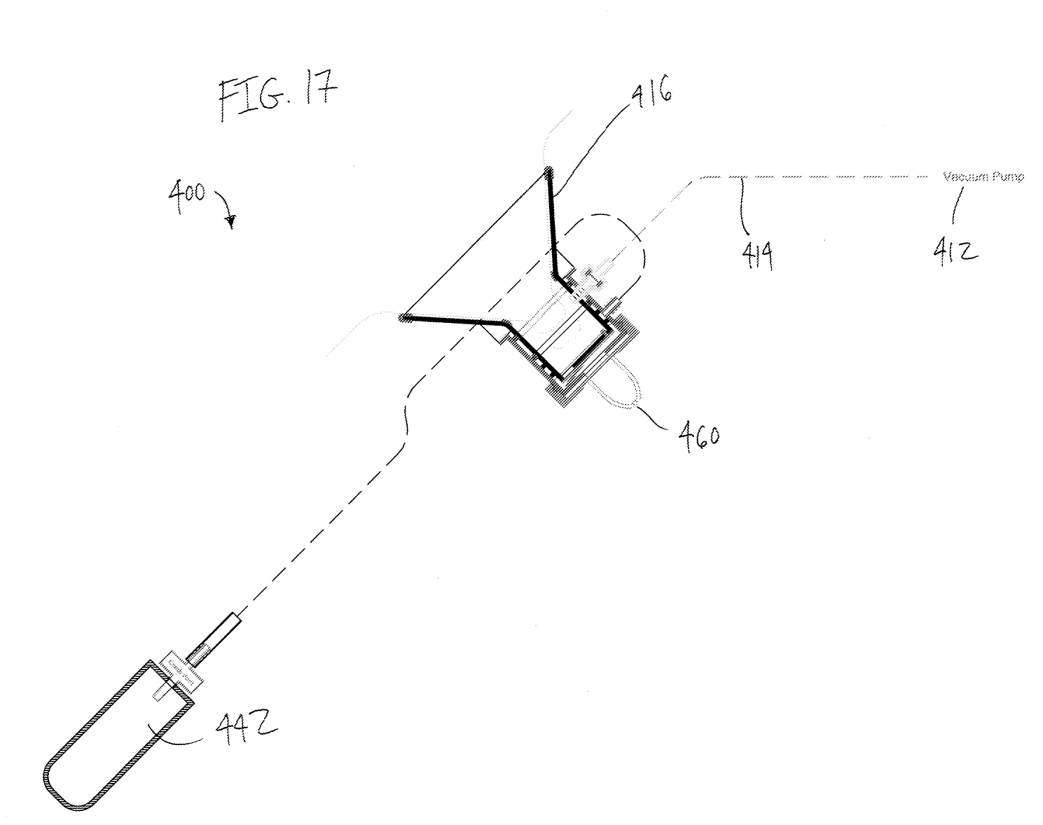

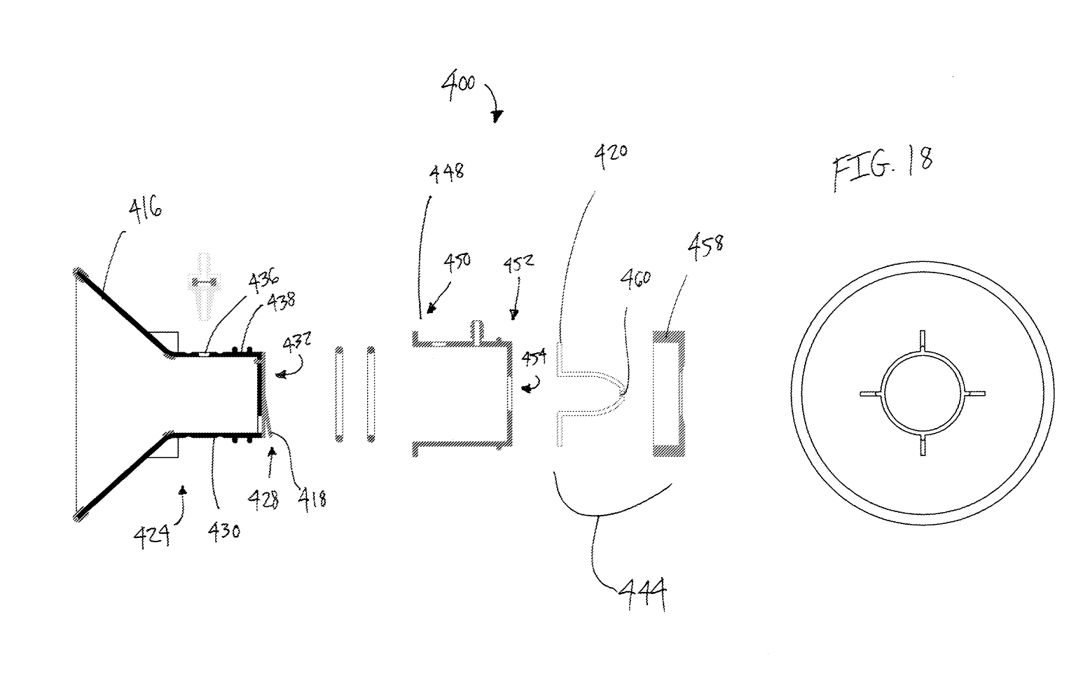

With reference to FIGS. 16-18, there is illustrated another exemplary breastfeeding assembly 400 according to an embodiment of the present disclosure. Breastfeeding assembly 400 is configured and dimensioned to be utilized for breastfeeding or the like. In general, assembly 400 is structurally and/or functionally similar to assembly 10 and/or 100/200/300 discussed above, with some differences.

### Maternal Chamber 424

In exemplary embodiments, the maternal chamber 424 includes an inner breastshield section 416 (e.g., inner breast cup member 416). The proximal end 426 of the breastshield 416 is open and fits over the breast. At the distal end 428 of the breastshield 416, the infant end, there is a tubular section or intermediate breastshield member/section 430 having an opening 432.

There is at least one hole 436 and/or 438 in the distal end 428. A hose barb and tubing 414 are inserted into this hole 436 to apply vacuum to the nipple using a vacuum source 412 via line 414. A protective membrane/filter 490 is seated between hole 436 and the tubing 414. A second hole 438 can be used to insert tubing into the fluid area of the assembly 400 to evacuate breast milk to a collection reservoir 442.

In another embodiment, the vacuum source 412 (e.g., miniature pump 412) is directly attached to the assembly 400, eliminating and/or reducing the need for tubing 414 in between the assembly 400 and vacuum source 412.

### Valve(s)

In exemplary embodiments, at least one valve member 418 (e.g., similar to valve member 218—diaphragm check valve or the like) allows for the anterograde flow of milk from the maternal chamber 424 to infant chamber 444 (e.g., only), as similarly described above.

### Chamber Connector 448

In exemplary embodiments, the chamber connector or outer breastshield member 448 attaches the maternal 424 and infant chambers 444, and allows for the passage of milk from the maternal 424 to the infant chamber 444. In general, the chamber connector 448 includes at least two areas—the neck 450 and the head 452.

The neck 450 fits over the distal end 428 of the maternal chamber 424. There can be two small holes in the neck 450.

In exemplary embodiments, the head 452 of the chamber connector 448 is configured and dimensioned to attach to the infant chamber 444. The head 452 has an opening 454 at the outer distal end to allow for the flow of milk into the infant chamber 444.

### Infant Chamber 444

In exemplary embodiments, the infant chamber 444 includes an outer member 420 (e.g., nipple member 420) and a securement member 458 (e.g., circular ring member 458). In certain embodiments, the ring 458 fits tightly over (e.g., threadably engaged with) the head 452 of the chamber connector 448, and securely and releasably attaches/secures the infant chamber 444 to the chamber connector 448.

There is at least one small opening 460 in the end of the nipple member 420 in the infant chamber 444, allowing for milk to flow out of the nipple member 420 and into the infant's mouth when sucking.

In alternative embodiments, it is noted that many of these parts/members can be combined/integral. For example and with reference to FIG. 18, with overmolding the breastshield straight/tubular portion 430 can be larger/longer and have compartments built into it, with an overmolded softer tip (e.g., 420) mounted at the end of portion 430, or portion 430 can have a hole for the rubber tip to be placed on the inside (to stick out). This could eliminate the need for securement member 458 and the chamber connector 448 as separate pieces.

It is noted that in certain embodiments, valve member 418 includes a flap or diaphragm that acts as a barrier to create vacuum. Instead of the flap/diaphragm, the fluid itself covering a hole can act as the barrier (e.g., if the suction is from the top contacting air and not contacting and pulling up fluid). The fluid is heavy enough to stay at the bottom of the chamber (424). In this regard, there could be an overflow port/opening or the like connected to the breastshield 416 and/or 430 to prevent fluid from rising too much. If a conventional flap/diaphragm (like on conventional pumps) is utilized, one should ensure that the flap is open on the distal portion (and mostly vertical), because it works with gravity.

It is noted that the baby could be able to practice the sucking (such as having the nipple member connected to a tube and to a reservoir). Even if the baby is not strong enough to pull milk out of the breast, one can gradually increase the resistance to make them suck harder to pull from the reservoir. In this regard, the flow control could be not only flow control of the pump, but a flow control to the baby via the valve/tubing/hole sizes, etc. Moreover, the artificial nipple could be completely enclosed and separate from the breastshield (e.g., the baby is further away from you) and connected by a tube to the breastshield, while still allowing the entire pumping/feeding to happen at the same time (e.g., for older babies). In this regard, if the nipple member is still attached to the whole assembly, one could have an inner tube, for the nipple member to reach the reservoir.

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

What is claimed is:

1. A breastfeeding assembly comprising:
a breastshield member configured and dimensioned to be positioned over a breast;
a nipple member configured and dimensioned to be mounted with respect to the breastshield member; and
a suction/vacuum source operatively connected to the breastshield member, the suction/vacuum source configured to provide vacuum or negative pressure to a maternal nipple of the breast through the breastshield member to facilitate the removal of milk for infant consumption from the breast during real-time breastfeeding.

2. The breastfeeding assembly of claim 1, wherein the suction/vacuum source is electric or manual.

3. The breastfeeding assembly of claim 1, wherein the nipple member is fabricated from a food grade polymer.

4. The breastfeeding assembly of claim 1, wherein the nipple member includes an attachment area for the infant's mouth that includes one or more openings, slits or holes for milk to flow therethrough.

5. The breastfeeding assembly of claim 1, wherein the suction/vacuum source is external to the breastshield member and is in communication with the breastshield member via tubing.

6. The breastfeeding assembly of claim 1, wherein the breastshield member includes one or more milk exit portals.

7. The breastfeeding assembly of claim 1 further comprising a flow control member operatively associated with the suction/vacuum source.

8. The breastfeeding assembly of claim 5, wherein the tubing is configured and dimensioned to attach to a breast pump.

9. The breastfeeding assembly of claim 1, wherein a distal end of the breastshield member is configured and dimensioned to allow the maternal nipple to move in a back and forth manner relative to the distal end of the breastshield member.

10. The breastfeeding assembly of claim 1, wherein the suction/vacuum source is configured to regulate and facilitate the control of milk flow.

11. The breastfeeding assembly of claim 10, wherein the suction/vacuum source is configured to be turned on and off to regulate and facilitate the control of milk flow.

12. The breastfeeding assembly of claim 5, wherein at least a portion of the breastshield member defines a vacuum compartment and at least a portion of the nipple member defines a milk reservoir compartment; and
wherein at least one valve member is positioned between the vacuum compartment and the milk reservoir compartment, the at least one valve member configured to intermittently seal the vacuum compartment to create vacuum that intermittently elongates the maternal nipple to release milk, and then allows milk to flow from the vacuum compartment to the milk reservoir compartment and not to the tubing.

13. The breastfeeding assembly of claim 12, wherein the at least one valve member is a one-way valve or a check valve positioned on a supporting surface of the breastshield member, the at least one valve member configured to provide for: (i) the substantially complete and intermittent sealing of the vacuum compartment, and (ii) the flow of milk from the vacuum compartment to the milk reservoir compartment and to the infant's mouth or to a collection reservoir.

14. The breastfeeding assembly of claim 5, wherein at least a portion of the tubing is positioned between the breastshield member and the nipple member.

15. The breastfeeding assembly of claim 12, further comprising a connector member, the connector member configured to releasably secure the breastshield member and the nipple member relative to one another.

16. The breastfeeding assembly of claim 15, further comprising a securement member, the securement member and the connector member configured to releasably secure the breastshield member and the nipple member relative to one another.

17. The breastfeeding assembly of claim 1, wherein the breastshield member and the nipple member are fabricated from food grade washable material.

18. The breastfeeding assembly of claim 1, wherein the breastshield member can be fabricated and user-selected in various sizes for maternal comfort.

19. The breastfeeding assembly of claim 1, wherein the nipple member can be fabricated and user-selected with various levels of rigidity to provide different milk flow rates to match infant feeding ability.

20. The breastfeeding assembly of claim 4, wherein the nipple member can be fabricated and user-selected with various sizes or shapes of the one or more openings, slits or holes to provide different milk flow rates to match infant feeding ability.

21. The breastfeeding assembly of claim 1, wherein tubing connects the milk reservoir compartment to an isolated collection reservoir, the tubing configured to collect milk overflow from the milk reservoir compartment and send it to the isolated collection reservoir for subsequent infant consumption.

22. A method for breastfeeding comprising:
providing a breastshield member;
mounting a nipple member with respect to the breastshield member;
operatively connecting a suction/vacuum source to the breastshield member;
positioning the breastshield member over a breast; and
providing vacuum or negative pressure via the suction/vacuum source to a maternal nipple of the breast through the breastshield member to facilitate the removal of milk for infant consumption from the breast during real-time breastfeeding.
